(19) United States
(12) Patent Application Publication
(10) Pub. No.: US 2009/0275929 A1
(43) Pub. Date: Nov. 5, 2009

(54) SYSTEM AND METHOD FOR CONTROLLING MEASUREMENT IN AN EYE DURING OPHTHALMIC PROCEDURE

(75) Inventor: Leander Zickler, Mountain View, CA (US)

Correspondence Address: ABBOTT MEDICAL OPTICS, INC., 1700 E. ST. ANDREW PLACE, SANTA ANA, CA, 92705, US

(73) Assignee: AMO Development, LLC, Santa Ana, CA (US)

(21) Appl. No.: 12/433,835

(22) Filed: Apr. 30, 2009

Related U.S. Application Data
(60) Provisional application No. 61/049,345, filed on Apr. 30, 2008.

(51) Int. Cl. A61F 9/008; A61B 3/113; A61B 3/10; A61B 18/20; A61B 3/14

(52) U.S. Cl. 606/5; 351/210; 351/246; 606/4; 351/206

(57) ABSTRACT

Systems and methods are provided for sensing a movement of an eye. The system includes a light source configured to illuminate the eye with a first light having a polarization state, an image capture apparatus configured to generate at least one image of the eye based on a reflected light from the eye, an analyzer optically coupled to the image capture apparatus and configured to filter a second light from the reflected light, and a processor coupled to the image capture apparatus and configured to determine the movement of the eye based on the at least one image of the eye. The reflected light is based on the first light, and the second light has the polarization state.

### CROSS-REFERENCES TO RELATED APPLICATIONS

This application claims the benefit of provisional application No. 61/049,345, filed on Apr. 30, 2008, the full disclosure of which is incorporated herein by reference.

### BACKGROUND OF THE INVENTION

1. Field of the Invention

The present invention relates generally to laser eye surgery systems and methods and more particularly, to systems and methods for controlling measurements in the eye during ophthalmic procedures.

2. Background

Laser-based systems have been used in ophthalmic surgery, such as on corneal tissues to correct vision defects. These systems use lasers to achieve a desired change in corneal shape. For example, the laser removes thin layers of corneal tissue using a technique generally described as ablative photodecomposition. Laser eye surgery techniques are useful in procedures such as photorefractive keratectomy, phototherapeutic keratectomy, laser in situ keratomileusis (LASIK), and the like.

Wavefront measurement systems have been utilized to measure the refractive characteristics of a particular patient's eye. An ablation pattern may thus be customized based on wavefront measurements to correct minor refractive errors and provide visual acuities of about 20/20 or greater than 20/20. However, these measurement systems are not immune from wavefront measurement error. Additionally, the calculation of the ablation profile, the transfer of information from the measurement system to the ablation system, and the operation of the ablation system may each introduce errors to the overall corrective procedure. The actual visual acuities provided by real-world wavefront-based correction systems may be less than theoretically possible.

The customized laser ablation pattern is preferably aligned and/or oriented with the patient's eye during the entire treatment. For proper registration between the wavefront measurement and the treatment to be delivered to the patient's eye (e.g., the customized laser ablation pattern), the wavefront measurement and the patient's eye should share a common coordinate system or reference system. For example, when the wavefront measurement is taken, the patient is typically seated. However, when the treatment (e.g., laser eye surgery to affect the customized laser ablation pattern) is performed, the patient is typically in a supine position, which may position the patient's eye in a different position (e.g., a different torsional orientation) than the position of the patient's eye during the wavefront measurement.

Moreover, even if the patient is positioned in the same initial position and/or torsional orientation, the eye often undergoes a cyclotorsional rotation. Improper compensation or lack of compensation for this cyclotorsional rotation will likely reduce the benefits of the refractive surgery, particularly in cases of astigmatism and other non-rotationally symmetric aberrations. The torsional movement of the patient's eye during the treatment may decrease the optimal delivery of the customized ablation pattern to the patient's eye.

Additionally, the ability to track or follow other movements of a patient's eye is recognized as a desirable feature in laser eye surgery systems. Movements of the eye include both voluntary movements and involuntary movements. For example, even when the patient is holding “steady” fixation on a visual target to minimize voluntary eye movement, involuntary eye movement may still occur. Tracking of the eye during laser eye surgery has been proposed to avoid uncomfortable devices which attempt to achieve total immobilization of the eye. Some eye-tracking techniques for use with laser eye surgery attempt to provide both lateral tracking (e.g., along an x-axis and a y-axis) and information regarding the position of the eye along the optical axis (e.g., along a z-axis).

A variety of eye tracking and beam scanning techniques have been proposed to align the customized laser ablation pattern with the eye. Some of these techniques rely upon imaging the eye. Specular reflections from natural (e.g., tear film, epithelium, stroma, sclera, and the like) or artificially created (e.g., from a corneal flap incision, surgical instrument, intraocular lens, implant, and the like) refractive index discontinuities of the human eye may decrease image detail, which would interfere with position detection and motion compensation. For example, following a corneal flap incision and lift, the stromal bed is revealed to receive the customized laser ablation pattern. In some cases, this stromal bed may create specular reflections that interfere with the imaging relied upon for position detection and motion compensation. The regions of specular reflections may be detected or a priori known and excluded from processing.

Accordingly, it would be desirable to provide improved laser eye surgery systems, devices, and methods that enhance the alignment of a desired ablation with the patient's eye. It would also be desirable to provide eye tracking and motion compensation for ophthalmic laser procedures with increased robustness and dynamic range of eye position detection. In particular, it would be desirable to provide improved laser eye surgery systems, devices, and methods that minimize interference with position detection and motion compensation. It would also be beneficial if these improvements provided enhanced tracking effectiveness and motion compensation and allowed the incorporation of such capabilities into known laser eye surgery systems without substantial modifications to the laser eye surgery system. Additionally, other desirable features and characteristics of the present invention will become apparent from the subsequent detailed description and the appended claims, taken in conjunction with the accompanying drawings and the foregoing technical field and background.

### SUMMARY OF THE INVENTION

The present invention is generally directed to ophthalmic devices, systems, and methods for laser eye surgery with an enhanced alignment of a desired ablation with the patient's eye. In general, a specular reflection tends to maintain the particular polarization, if any, of the source of light, whereas a diffuse reflection tends to have randomly polarized light. By directing polarized illumination (e.g., having a pre-determined polarized state) at the eye and detecting the position of the eye or movement of the eye based on the diffuse reflected light from the eye, interference normally associated with specular reflections (e.g., associated with tear film, epithelium, stroma, sclera, flap incision, surgical instrument, intraocular lens, implant, and the like) is efficiently suppressed resulting in an increased robustness and dynamic range of eye position detection and/or eye movement detection.

In one embodiment, a system is provided for sensing a movement of an eye. The system includes a light source configured to illuminate the eye with a first light having a polarization state, an image capture apparatus configured to generate at least one image of the eye based on a reflected light from the eye, an analyzer optically coupled to the image capture apparatus and configured to filter a second light from the reflected light, and a processor coupled to the image capture apparatus and configured to determine the movement of the eye based on the at least one image of the eye. The reflected light is based on the first light, and the second light has the polarization state.

In another embodiment, system is provided for ablating a cornea of an eye with a laser treatment. The system includes a laser assembly configured to output a pulsed laser beam, an image capture apparatus comprising a light source configured to illuminate the eye with a first light having a polarization state, an analyzer optically coupled to the image capture apparatus and configured to filter a second light from the reflected light, and a controller coupled to the laser assembly and the image capture apparatus. The image capture apparatus is configured to generate at least one image of the eye based on a reflected light from the eye, and the reflected light is based on the first light. The second light has the polarization state. The controller configured to determine a movement of the eye based on the at least one image of the eye and direct the laser assembly to deflect the pulsed laser beam in correlation with the movement of the eye.

In another embodiment, a system is provided for ablating a cornea of an eye with a laser treatment. The laser treatment is generated in association with a first image of the eye. The system includes a laser assembly configured to output a pulsed laser beam, an image capture apparatus comprising a light source configured to illuminate the eye with a first light having a polarization state, an analyzer optically coupled to the image capture apparatus and configured to filter a second light from the reflected light, and a controller coupled to the laser assembly and the image capture apparatus. The image capture apparatus is configured to generate a second image of the eye based on a reflected light from the eye, and the reflected light is based on the first light. The second light has the polarization state. The controller is configured to register the first image and the second image, align the laser treatment with the second image of the eye, and direct the laser assembly to output the pulsed laser beam at the cornea in correlation with the laser treatment.

In another embodiment, a method of modifying a refractive profile of an eye with a laser treatment is provided. The method includes illuminating the eye with a first light having a polarized state, filtering a second light from a reflected light from the eye, capturing at least one image of the eye based on the reflected light from the eye, determining a position of the eye based on the at least one image of the eye, aligning a laser treatment based on the position of the eye, and directing a pulsed laser beam at a corneal surface of the eye in correlation with the laser treatment. The reflected light is based on the first light, and the second light has the polarized state.

The present invention is particularly useful for enhancing the accuracy and efficacy of laser eye surgical procedures such as photorefractive keratectomy (PRK), phototherapeutic keratectomy (PTK), laser in situ keratomileusis (LASIK), and the like. The efficacy of the laser eye surgical procedures can be enhanced by tracking the lateral movement and torsional orientation of the patient's eye so that a laser ablation pattern is more accurately aligned with the real-time orientation of the patient's eye.

While the system and methods of the present invention are described primarily in the context of improving a laser eye surgery system, it should be understood the techniques of the present invention may be adapted for use in alternative eye treatment procedures and systems such as femtosecond lasers and laser treatment, infrared lasers and laser treatments, radial keratotomy (RK), scleral bands, follow up diagnostic procedures, and the like.]

### BRIEF DESCRIPTION OF THE DRAWINGS

In the drawings, wherein like reference numerals refer to similar components:

FIG. 1 is a block diagram of an ophthalmic laser surgery system in accordance with one embodiment;

FIG. 2 is a block diagram of the system shown in FIG. 1 illustrating a path of the laser energy pulse propagating therethrough and a laser control path in accordance with one embodiment;

FIG. 3 is a block diagram of the system shown in FIG. 1 illustrating an optical path from the depth ranging and tracking assembly and a depth position control path in accordance with one embodiment;

FIG. 4 is a block diagram of the system shown in FIG. 1 illustrating an optical path from the parallax depth ranging assembly to the eye and an imaging control path in accordance with one embodiment;

FIG. 5 is a block diagram of the system shown in FIG. 1 illustrating an optical path and control loops for the X-Y place tracking means in accordance with one embodiment;

FIG. 6 is a front view of the iris incident on two quadrant detectors of a spatially sensitive sensor for X-Y tracking in accordance with one embodiment;

FIG. 7 is a block diagram of an ophthalmic laser surgery system in accordance with another embodiment;

FIG. 8 is a perspective view of the laser delivery optics and tracking imaging axes of the system shown in FIG. 7;

FIG. 9 is a schematic diagram illustrating a camera position of the system shown in FIG. 7 for sensing movement of the eye along an X axis of the eye;

FIG. 10 is a block diagram of the system shown in FIG. 1 illustrating an interplay of the imaging assembly with the user interface;

FIG. 11 is a block diagram of the system shown in FIG. 1 illustrating optical paths between the topography assembly and the eye together with a topography control loop;

FIG. 12 is an image of the eye without polarized illumination;

FIG. 13 is an image of a corneal flap bed of the eye without polarized illumination; and

FIG. 13 is an image of the eye with polarized illumination in accordance with one embodiment.

### DETAILED DESCRIPTION

The present invention generally provides systems and methods for improving eye tracking and motion compensation during an ophthalmic laser procedure. Polarized illumination is directed at the patient's eye, and reflected diffuse light having random polarization state is detected for eye tracking and motion compensation (e.g., lateral movements of the eye, cyclotorsional movements, and the like). Interference that may arise from specular reflections is efficiently suppressed (e.g., by filtering out light having the polarization state of the polarized illumination) resulting in an increased robustness and dynamic range of eye position detection. In one embodiment, where eye tracking and motion compensation is based on image capture, one or more analyzers (e.g., filters) are utilized to filter light having the polarization state of the polarized illumination from the light received during image capture.

The eye tracking and motion compensation is used to stabilize the motion of the patient (e.g., the patient's eye). An automated target acquisition and tracking system allows a surgeon/user to predetermine a firing pattern (e.g., laser treatment) based on an image that is automatically stabilized over time. The systems and methods disclosed herein incorporate a mapping and topography means for reconstructing the corneal surface shape and thickness across the entire cornea. It is furthermore within the scope of the present invention to provide such global measurements of the corneal refractive power without sacrificing local accuracies and while maintaining sufficient working distance between the eye and the front optical element of the instrument (e.g., a front objective lens). These measurements may be executed on-line within time scales that are not limited to human response times.

In one embodiment of the present invention, the corneal refractive power may be measured using a unique projection and profilometry technique coupled with signal enhancement methods for surface reconstruction as disclosed by U.S. Pat. No. 5,170,193 and further extended in larger corneal cross-sections via techniques described in U.S. Pat. No. 5,283,598, both incorporated herein by reference. In another embodiment, digitized slit lamp video images are used to measure the local radii of curvature across the entire corneal surface as well as the thickness of the cornea, with no built-in a-priori assumptions about the corneal shape.

While tissue topography measures parameters instrumental to defining templates for the surgery (e.g., refractive power), additional mapping and imaging means may be incorporated to allow reliable, on-line monitoring of a given surgical procedure. The imaging means is intended to record, in three-dimensions, the location of significant features of the tissue to be operated upon, including features located well within the subject tissue. Continuously updated video images may be presented to the surgeon/user as the surgery progresses, and these images can be produced in a cost effective manner with high resolution and high magnification across a large field of view and at sufficiently low illumination levels to prevent any discomfort to the patient.

Movements of the eye are followed by the tracking system with closed-loop refresh processing rates surpassing those achievable by unaided human inspection. Tracking by following the subject eye tissue (i.e., recognizing new locations of the same tissue and readjusting the imaging system and the surgical laser aim to the new location) assures that the laser, when firing through a prescribed pattern, follows a proscribed pattern within acceptable tolerances. For example, one tolerance is preferably within 5 microns in all situations during ophthalmic surgery. It is possible that with future use and experimentation, more stringent or alternatively more lax displacement error tolerances are desirable to improve overall system performance.

Stabilization of a moving target typically includes defining the target (e.g., the eye), characterizing the motion of the target, and repeatedly readjusting the aim of the apparatus or system in a closed-loop system. In one embodiment, the tracking information is obtained through means (e.g., indicia or landmarks) contiguous to the target region, which is mechanically and structurally considered as a part of the cornea. These means are unlikely to be affected by the course of the surgery and can provide a significant representation of non-surgically induced displacements. Involuntary motions of the eye (such as are caused by blood vessel pulsing) are thus accurately accommodated.

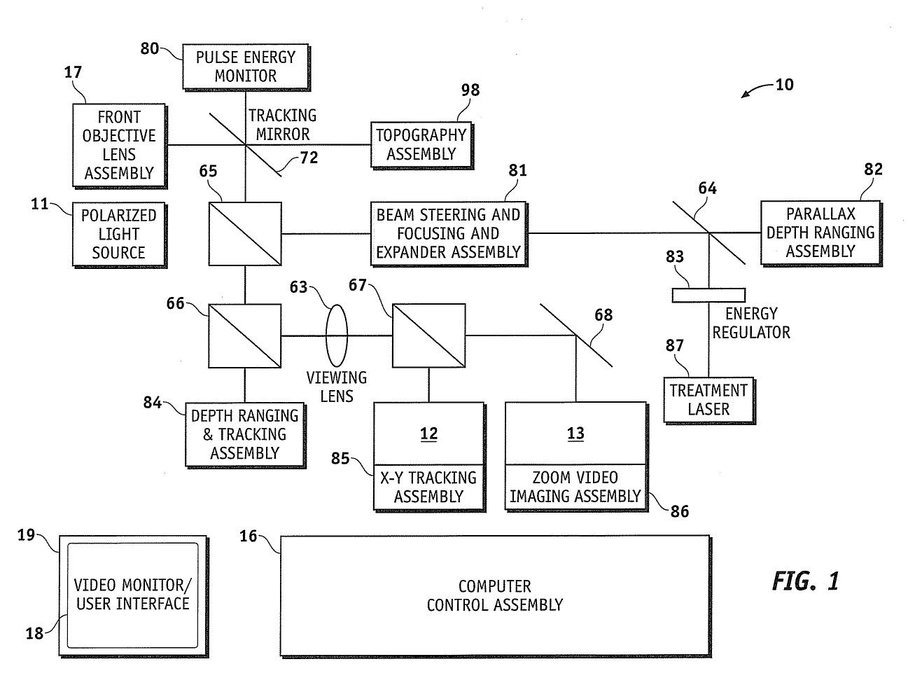

Referring to the drawings, an ophthalmic laser surgery system 10 is shown in FIG. 1 in accordance with one embodiment. The system 10 includes a user interface 19, a treatment laser 87, a computer control assembly 16 for a vision system and for firing the treatment laser 87, a polarized light source 11 for selectively illuminating a patient's eye, a final objective lens assembly or focusing lens or front lens 17 (e.g., an element of a microscope assembly, which may use one or more optical devices), through which images are taken and through which the laser beam is directed at the patient's eye (e.g., corneal tissue), a zoom video imaging assembly 86 (e.g., serving as a surgical microscope), an X-Y tracking assembly 85, a depth ranging and tracking assembly 84, a parallax depth ranging assembly 82, an energy regulator 83, a pulse energy monitor 80, various illuminators, a beam steering and focusing assembly 81, and a topography assembly 98. In this embodiment, all of these assemblies share an optical path defined by a final tracking mirror 72 and the front lens 17. Other elements (not shown) of the system 10 may include a support station housing the user interface 19, power supplies, a fire control/safety switch, other accessories for the system 10, and the like.

The computer control assembly 16 enables a surgeon/user to survey the topography and internal features of the tissue to be operated upon (e.g., an eye), via the user interface 19, and precisely control the timing as well as the direction, depth and spatial pattern of firing a laser beam in three-dimensions. The surgeon may control the firing of the laser 87 with “templates” which can be superimposed over an image of the tissue being operated upon, and which enable an automatic tracing of a desired laser firing pattern (e.g., a laser treatment) based upon prior experience or a surgeon's insights with similar surgical procedures. The templates may be pre-programmed or generated anew for each patient.

In one embodiment, an axial illuminating light beam is projected at the eye through the topography assembly 98 and the front lens 17. An off-axis slit illuminator (not shown), providing a ribbon-shaped illuminating light beam, may be used to augment and/or replace the axial illumination technique (e.g., such as described in Howland et al. “Noninvasive Assessment of the Visual System Topical Meeting,” Santa Fe, Feb. 4-7, 1991) depending on the particular kind of surgical procedure or error tolerances required thereof.

The tracking mirror 72 is in the path of light (whether transmitted or reflected) generated and/or received by all the various subassemblies of the system 10. In various embodiments, the tracking mirror 72 is driven piezoelectrically, electromagnetically, or through other means. A piezoelectric driver uses the change in shape of a quartz crystal in response to an electric current to move the mirror. An electromagnetic driver uses a coil of wire in a magnetic field which is made to move by passing an electric current through the coil. The electromagnetic driver is similar in function to a voice coil of an audio speaker. For either driver, the speed or acceleration of the entire tracking system is generally limited by the response of the drivers and the moment of inertia associated with the tracking mirror 72.

Combinations of various elements and/or subassemblies of the system 10 perform a number of different functions. For example, to identify a location to be treated on or in the cornea, the surgeon/user uses a combination of video imaging and automated diagnostic devices (e.g., such as described in U.S. Pat. No. 5,098,426 and U.S. patent application Ser. No. 475,657 (now abandoned)), depth ranging techniques (e.g., such as described in U.S. Pat. No. 5,162,641), surface topographical techniques (e.g., such as described in U.S. Pat. No. 5,054,907) together with signal enhancement techniques for obtaining curvatures and charting the contours of the corneal surface (e.g., such as described by U.S. Pat. No. 5,170,193), profilometry methods (e.g., such as disclosed in U.S. Pat. No. 5,283,598), image stabilization techniques (e.g., such as described by U.S. Pat. No. 5,162,641), which may all be combined together (e.g., such as using techniques described by U.S. Pat. No. 5,098,426 and U.S. patent application Ser. No. 475,657 (now abandoned)). U.S. Pat. No. 6,913,603 describes an integrated laser system combining one or more of the features described in the foregoing. All of the above listed patents, patent applications, and U.S. Pat. No. 5,391,165 are herein incorporated in entirety by reference.

One topography technique requires establishing the distance from the surface to be measured to the appropriate principal line of the front focusing lens. While there are several methods for establishing this distance, the modified confocal technique described by U.S. Pat. No. 5,283,598 represents one embodiment of such a measuring technique. During surgery or treatment, the target is live tissue, which is typically in continuous motion. The surface (e.g., to be measured by way of the topography assembly 98) preferably remains stable with respect to the measuring sensors located within topography assembly 98, zoom video assembly 86, and the known focal point of laser 87. In one embodiment, the position of the front lens 17 is continuously adjusted along the axial direction (e.g., as further described in U.S. Pat. No. 5,283,598).

A variety of structures and techniques may be used for both tracking of eye movements and scanning of a laser beam across the corneal tissue. An exemplary linear array eye-tracking system and method are described in co-pending U.S. Pat. No. 6,283,954, which is incorporated herein in entirety by reference. Other systems for tracking movement of an eye, particularly for use in laser eye surgery, are described in U.S. Pat. Nos. 5,865,832; 5,632,742; and 4,848,340, the full disclosures of which are also incorporated herein by reference. An exemplary “offset imaging” scanning system for selective ablation and sculpting of corneal tissue is described in European Patent Application Publication No. 628298, the full disclosure of which is hereby incorporated by reference. This offset imaging system allows a laser beam to be accurately directed on to a surface of the corneal tissue to mitigate myopia, hyperopia, astigmatism, or combinations of these ocular defects, particularly when the scanning or offset imaging system is combined with one or more variable apertures for profiling the laser beam. As described in co-pending U.S. patent Ser. No. 09/274,499, filed on Mar. 23, 1999, entitled Multiple Beam Sculpting System and Method (the disclosure of which is incorporated herein by reference), the laser beam may ideally be separated into a plurality of beamlets to minimize discontinuities adjacent the ablation edges. Alternative scanning systems are described in the following U.S. patents, which are also incorporated herein by reference: U.S. Pat. Nos. 5,556,395; 5,683,379; 5,391,165; and 5,637,109.

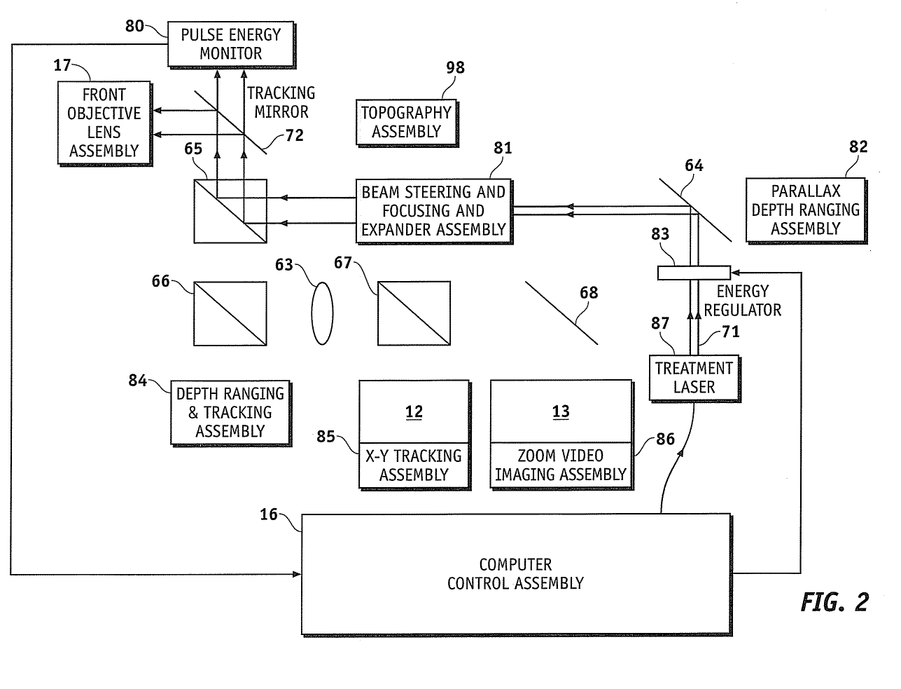

FIG. 2 shows a light path 71 as a laser beam emerges from laser 87, passes through the energy regulator 83, is expanded and directed in the beam steering and focusing assembly 81 (e.g., such as described by U.S. Pat. No. 5,391,165), and is aimed via tracking mirror 72 through front lens 17 onto the prescribed target site (e.g., the corneal tissue). In one embodiment, the tracking mirror 72 has an optical coating permitting a small portion of the laser energy to continue through tracking mirror 72, along path 73 to be detected by the pulse energy monitor 80, as depicted in FIG. 2.

The pulse energy sensed in the pulse energy monitor 80 is electronically relayed to the computer control assembly 16, which in turn analyzes the output energy from laser 87 and adjusts the proportion of laser energy of subsequent laser pulses to pass through the energy regulator 83. In one embodiment, the energy regulator 83 is a polarizer adjusted to be “crossed” with a polarized laser pulse, preceded by a rotatable half-wave retardation plate (not shown). The energy monitor 80 includes an integrating sphere and detector which can record energy on a pulse-by-pulse basis. For example, the detector calculates weighted exponential moving averages (e.g., of pulse energy), modified with a weighting factor, as well as the rate of change of the running average. The accuracy of measurement of the pulse energy is within about 5%, based on a calibration against conventional energy meters.

In one embodiment, the beam steering and focusing assembly 81 includes a beam expander 22 that provides depth of focus variations through collimation changes, and a dual set of Risley prisms (also known as Herschel prisms) 21 to steer and aim the beam (e.g., such as described in the U.S. Pat. No. 5,391,165). The beam expander 22 may have a set of lenses, a stepper motor, and a slide with about 75 mm travel corresponding to about 25 mm in the eye. Beam focus accuracy to within about 10 microns can be provided in this embodiment, based on standard optical components. The Risley prisms 21 have a lower moment of inertia and shorter lever arm as compared to alternatives, such as gimbaled mirrors. Faster aiming may be achieved with the lower moment of inertia, which may be enhanced using cylindrical coordinates, while the shorter lever arm permits aiming further off-axis without beam-clipping (e.g., vignetting) at the aperture of the front lens 17.

The surgical laser 87 emits radiation in the visible wavelength range to take advantage of the transmission properties of visible light in the optically clear tissues of the human eye. A frequency doubled Nd:YAG laser producing sufficiently short duration pulses (e.g., shorter than a few hundred nanoseconds, and preferably shorter than 10 nanoseconds) is preferably used to limit the amount of energy for ionizing material. In other embodiments, the laser 87 may be one of several types of flashlamp- or diode-pumped solid-state lasers (such as, Nd:YAG, Nd:YLF, HoYLF, Er:YAG, alexandrite, Ti:sapphire, or others) operating in the fundamental mode or a frequency-multiplied mode, a semiconductor laser, or an argon, excimer, nitrogen, dye, or any of a host of different laser, or combinations thereof, currently available or in development. The front lens 17 may be a quartz and magnesium fluoride focusing element to accommodate ultraviolet lasers (e.g., excimer lasers or frequency shifted solid-state lasers). The laser 87 preferably produces a pulsed beam that is controllable at least as to the level of energy per pulse, pulse peak power, and repetition rate. For ophthalmic applications that do not seek to generate laser lesions below the anterior surface of the cornea, or wherever incising the eye is an acceptable option as a preliminary or as part of the procedure, then excimer lasers, holmium lasers, carbon dioxide lasers, or some other ultraviolet or infrared laser may be an acceptable modality. In one embodiment, the surgeon is not restricted to surface effects or to incising the eye. With the same visible wavelength laser (for example, a frequency doubled Nd:YAG), the surgeon can select any tissue depth (whether on the corneal surface or below, whether on the posterior lens capsule or in the lens nucleus) for generating an effect without the necessity of exchanging laser modalities for different eye segments, provided there remains an optically clear path to the targeted layer in the corresponding visible range.

In the event of a non-visible-wavelength laser beam is used (e.g., strictly for ablating the front surface of the cornea, or strictly for coagulating blood vessels in the retina, or strictly for photodisrupting membranes on the posterior capsule), some variations in the optical configuration of system 10 will likely be required.

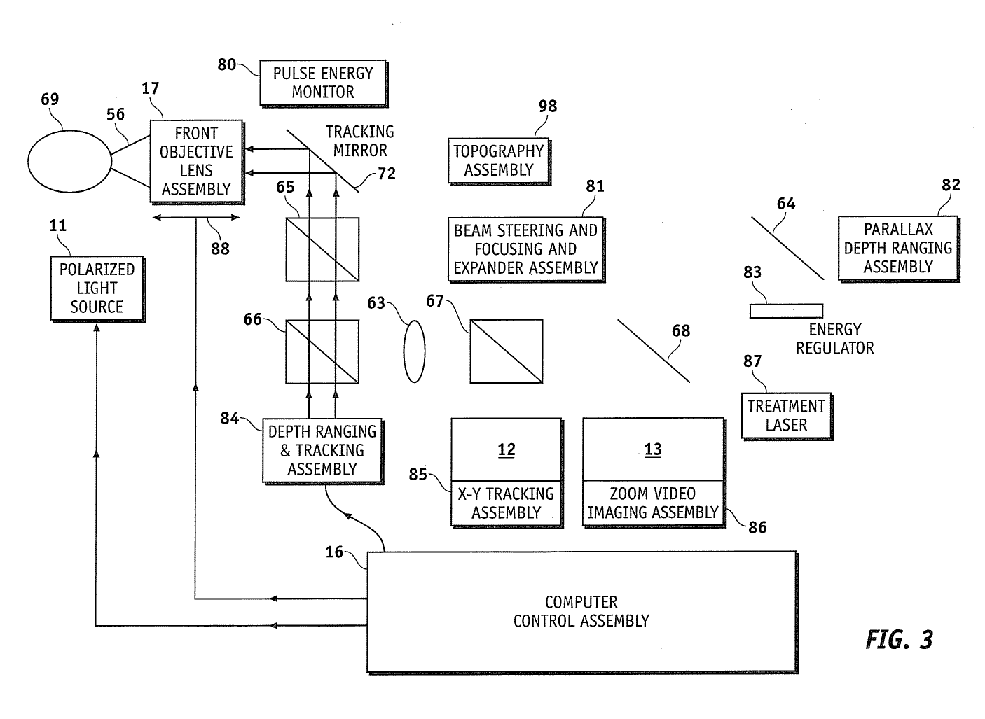

FIG. 3 shows an information path 88 for the depth ranging and tracking assembly 84. The depth ranging and tracking assembly 84 measures the distance from front lens 17 to the surface of an eye 69 and continuously adjusts the position of the front lens 17 along the path 88. In one embodiment, the path length 88 over which the front lens 17 is adjusted is about 5 mm. The depth ranging and tracking subassembly 84 together with the front lens 17 and the intervening optics maybe collectively be referred to herein as a confocal microscope. The confocal microscope uses optical elements in common with other equipment of system 10, namely the tracking mirror 72 and beam splitters 65 and 66. The front lens 17 is adjusted to focus, along a Z-axis, in response to shifts in the depth of the subject tissue feature, so that the system 10 returns to a focus on the corneal vertex 56 (e.g., the corneal region closest to the front lens 17).

Included in the depth ranging and tracking assembly 84 are depth tracking or “Z-axis” tracking sensors that detect a change in location of the eye 69 (e.g., such as described in U.S. Pat. No. 5,283,598) and relay the information to the computer control assembly 16, which computes a new desired position for the front lens 17 and issues instructions to a motor drive to relocate the front lens 17 to the new desired location. A closed-loop control is thus described which incorporates the real-time movements of the eye 69 within the decision process of adjusting the front lens 17 to within pre-determined tolerances. In one embodiment, the capture range for axial acquisition is within about +/−0.2 mm, and tracking rates in excess of about 40 Hz are within the closed-loop control capability for maximum ranges on the order of about 2 mm.

Since mirrors 64, 68, and 72 together with beam splitting cubes 65, 66 and 67 optically link the other assemblies of system 10 into a common axial path passing through the front lens 17, these components can all be referenced to the front lens 17 as if the distance between front lens 17 and eye 69 remains substantially constant. The surgeon is not required to continuously monitor eye movement for verifing a constantly changing focal position within the patient's eye.

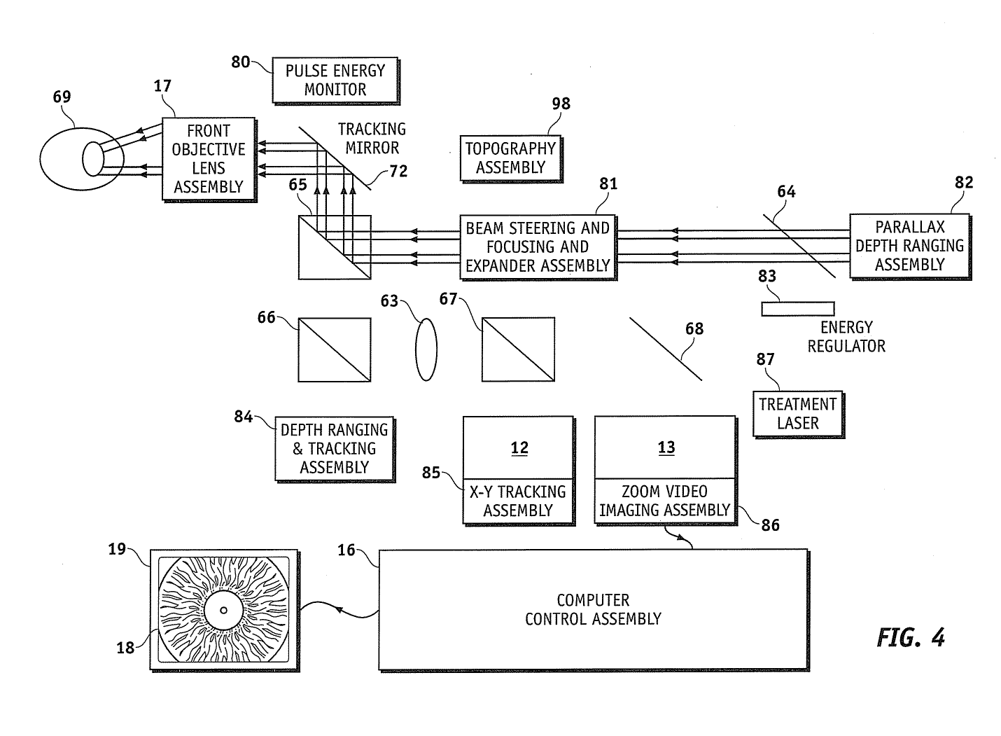

For procedures where the targeted tissue layers lie posterior to the cornea, the surgeon/user can use the parallax depth ranging assembly 82, as shown in FIG. 4. This assembly 82 relies on the intersection of two beams of light (from, e.g., a He—Ne illuminator laser) converging to a common point on a given surface. In one embodiment, the parallax depth ranging assembly 82 allows mapping of a mesh of points, acquired through judicious adjustment of a zoom camera to a short depth-of-focus (maximum magnification), which along with corresponding variation of the focus on the parallax depth ranging assembly 82, produces a series of diffraction limited spots on the structures behind the cornea (e.g., iris, lens, etc.). In this manner, the resulting surface defines a desired template.

The inclusion of the parallax depth ranging assembly 82 within the system 10 overcomes some difficulties commonly associated with specular reflection techniques used for detection of the location and measurement of ocular features. In general, the tear surface layer overlying the corneal surface epithelium is usually detectable and measurable by specular light reflection techniques. The reflected light signal is generally insufficient for extracting topographic information about the endothelium surface of the cornea (e.g., less than about 0.02% reflection versus about 4% reflection from the epithelium) as well as for characterizing the three-dimensional shape of the anterior and posterior capsules of the crystalline lens of the eye 69. The parallax depth ranging assembly 82 provides the option of using a combination of standard techniques, which rely on images of a target site, to determine, within the inherent error tolerances of the technique, when the system 10 is focused on a desired surface. The precise focal point of the beam can then be varied by altering the incoming beam divergence by way of defocusing the beam expander 22 (e.g., within the beam steering and focusing assembly 81). By redefining the origin of a given procedure to coincide with the depth at which the parallax depth ranging assembly 82 is focused on a surface, this new identified surface becomes the reference surface for performing a surgical procedure. Via the user interface 19 (e.g., such as described in U.S. Pat. No. 5,098,426 and/or U.S. patent application Ser. No. 475,657), the surgeon/user can define lesion templates or configurations to be performed at a given depth with respect to the new identified surface.

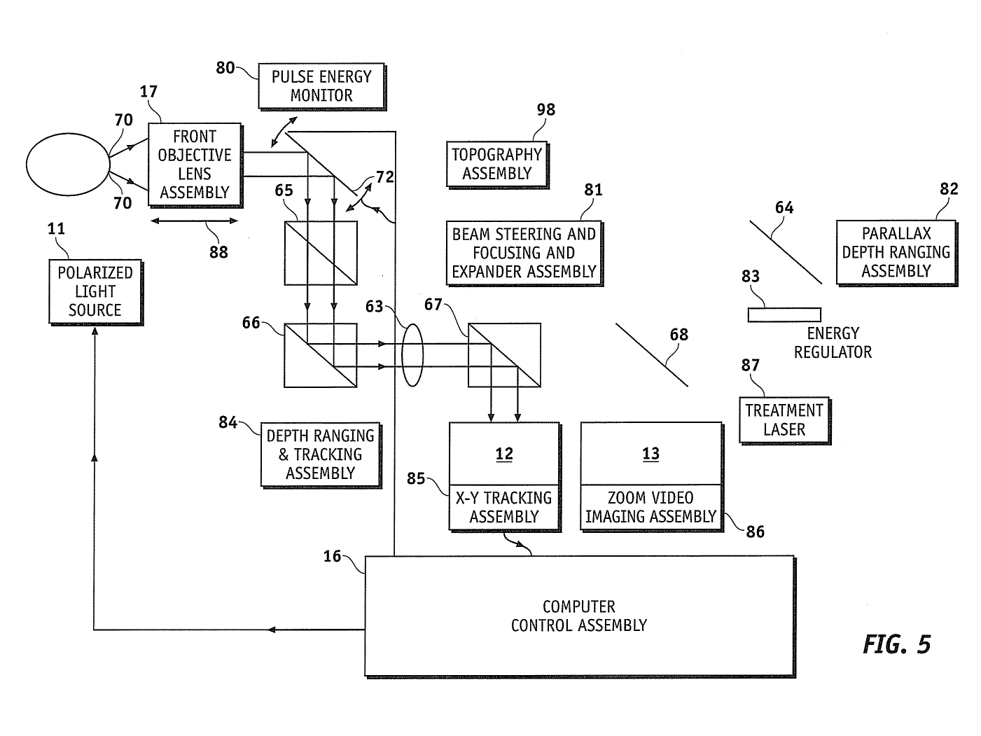

Similarly, the motion of the eye 69 along a plane orthogonal to the Z-axis associated with the front lens 17 can also be stabilized. This is achieved using the X-Y tracking assembly 85 and the polarized light source 11 (shown in FIG. 5) in accordance with one embodiment. To define the location of a tissue layer within the eye that is capable of movement, a tracking landmark is preferably located contiguous with the targeted tissue, and this landmark should mechanically behave (e.g., move) in a manner similar to the targeted issue. For corneal refractive surgery, the limbus of the eye, at the outermost radial edge of the cornea, can be used as a tracking landmark. In effect, pursuing the motions of the limbus allows the system 10 to replicate the template pattern presented on the display by the user interface 19, even though the eye surface may appreciably deform during the course of the surgical procedure.

In one embodiment, the X-Y tracking assembly 85 includes high speed quadrant detectors and a microprocessor such that updated position information is fed to the tracking mirror 72 at frequencies substantially greater than the repetition rate of the laser 87, or the frame rate of the imaging camera associated with the zoom video imaging assembly 86. The response time of the quadrant detectors and processor are preferably sufficiently faster than the maximum repetition rate of the laser 87 (e.g., for disabling laser firing, if necessary). The response time of the quadrant detector and processor is also preferably higher than that of the tracking mirror 72, and the tracking mirror 72 which should be capable of sufficiently high acceleration and velocity to compensate for the fastest motion possible by the intended target (e.g., the eye movement).

In FIG. 5, the light source 11 may be activated to illuminate the eye with polarized light (e.g., having a pre-determined polarization state). Light from limbus 70 (e.g., derived from the polarized light) passes through the front lens 17, is reflected by the tracking mirror 72, and is propagated via the beam splitting cubes 65, 66 through a viewing lens 63 for reflection off a beam splitter 67 to sensors of the X-Y tracking assembly 85. In this embodiment, the X-Y tracking assembly 85 includes an analyzer 12 for filtering light having the pre-determined polarization state from the light introduced to the X-Y tracking assembly 85. In other embodiments, the analyzer 12 is a separate optical device that may be selectively interposed along the optical path from the beam splitter 67 to the X-Y tracking assembly 85 when the light source 11 is activated. Specular reflection from the eye generally maintains polarization (e.g., the pre-determined polarization state of the light from the light source 11), whereas diffuse reflection from the eye generally has a random polarization. The analyzer 12 filters out light having the pre-determined polarization state and thus, the analyzer 12 removes specular reflections from the images analyzed by the X-Y tracking assembly 85.

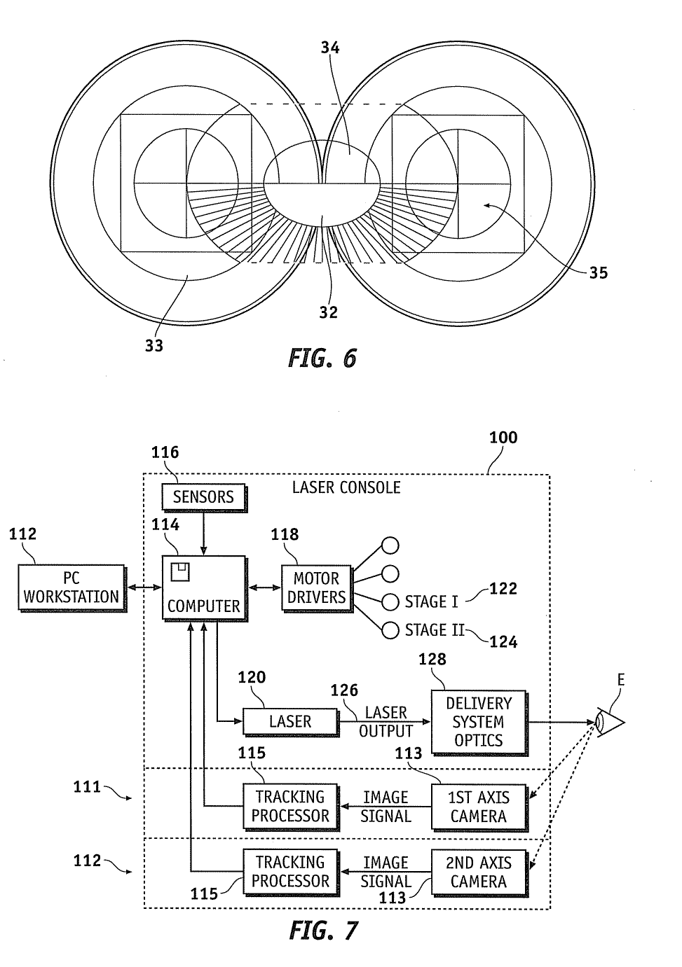

In one embodiment, the X-Y tracking assembly 85 includes a spatially sensitive sensor having two quadrant detectors to track an image of the outer rim of the iris 32 (e.g., at the limbus) or other tracking landmark on the eye. As shown in FIG. 6, the image of quadrant detectors (each with four quadrants 35, in this example) has a bright field corresponding to an image of the sclera 33, adjacent to another field representing an image of the iris 32. A central core is an image of the pupil 34 and is not captured by the quadrant detectors, as FIG. 6 illustrates, leaving a single sharp boundary to track. With various cells of the quadrant detector connected through differential amplifiers and normalized by a sum, the resultant signals are sensitive to the position of a centroid of illumination for any of the above patterns. The quadrant detectors integrate the image illumination striking each quarter of the detector face. The luminosity impingent on the detector faces then generate voltage differences corresponding to the integrated differences in light hitting the detector regions. A change in the background light intensity can typically be ignored because the increase across each of the four (or eight) quadrants 35 of the detector face remains substantially the same. Voltage sums and differences among the quadrants serve to establish the relative direction of motion between two contiguous readings of the limbus position. A shift in intensity at the sensor is thereby traced to a motion of the limbus. These dedicated quadrant detectors rapidly record voltage changes and can quickly observe and quantify contrast changes and edge motions (e.g., less than about 100 ms). In other embodiments, similarly fast but more sensitive position sensing detectors may be used yielding enhanced performance at even lower light levels.

Information representing the voltage change is relayed to the computer control assembly 16 where the actual coordinate shift is calculated. The computer control assembly 16 then determines the angular corrections to be relayed to the tracking mirror 72. For example, a voice coil or other electromagnetic drive assembly to pivot the orientation of mirror 72 can be activated to stabilize the X-Y motion of the limbus with respect to system 10.

In one embodiment, the range of use, or travel, is about 2 mm in the X-Y plane. For ophthalmic applications, where the principal motions of the eye are rotations, the range of use may be referenced in terms of angular sweep of the eye. For example, an angular motion of the eye of about 5° (e.g., deviation from the optical axis) is well within the operation capabilities of the X-Y tracking assembly 85. For a sighted human patient, it has been estimated that such range of use can acquire an eye looking at an image point located in the far field (relative to the patient) and situated along the optical axis of the apparatus. The transducers of the X-Y tracking assembly 85 adjust the position of the tracking mirror 72 along two pivotal axes at accelerations on the target in excess of about 20 microns/ms for full amplitudes greater than about 2 mm, based on microprocessor-provided information relating to the new location of the same tissue.

The surface of the eye may be displaced in translational and/or by rotational motions centered on the globe of the eye. Because the tracking mirror 72 pivots about a point (e.g., within its assembly) that may be different from the eye's center of rotation, a desired change in the position of the tracking mirror 72 may require a correction of the X-Y axis position of the depth ranging and tracking assembly 84. A compensating mirror (not shown) within the depth ranging and tracking assembly 84 may be used for this correction.

The X-Y tracking assembly 85 has the advantage of being able to find an absolute position of the target even after a temporary loss of tracking. For example, if a surgical procedure is in process and an obstacle, such as a blinking eyelid in many ophthalmic procedures, interferes with the tracking image such that the procedure is interrupted or temporarily aborted, the X-Y tracking assembly 85 automatically stores (e.g., in memory) the last position in the firing sequence. Once the target is reacquired, the location of the next point in the firing sequence can be automatically determined and the tracking mirror 72 can be repositioned accordingly.

FIG. 7 is a block diagram of an opthalmological surgery system 100 in accordance with another embodiment. In this embodiment, two off-axis image capture devices are used, with each image capture device sensing movement of the eye along an associated lateral eye movement axis. The image capture devices, sometimes referred to herein as cameras, are typically disposed off of the optical axis of the eye, which is often (but not necessarily) co-axial with the treatment axis of the laser system (e.g., the Z-axis). The lateral movements of the eye tracked by the two off-axis camera system 100 will often be described with reference to horizontal and vertical motions. As used herein, horizontal motions are from right to left or left to right relative to the patient, and vertical motions are along the inferior/superior orientation relative to the patient. The first and second motion axes associated with the first and second image capture devices need not necessarily be orthogonal. Even when these motion axes are orthogonal (such as when they define orthogonal X and Y lateral orientations), the axes need not necessarily be aligned with the horizontal and vertical orientations.

In this embodiment, the system 100 includes horizontal and vertical trackers 111, 112, which function in place of the X-Y tracking assembly 85 shown in FIGS. 1-5. Each of trackers 111, 112 includes a camera 113 and an associated tracking processor 115. The system 100 also includes the laser 87 which generates a laser beam 126, which is selectively directed toward the eye, E, by delivery system optics 128 (e.g., such as the front lens 17, tracking mirror 72, beam splitter 65, and mirror 64 shown in FIGS. 1-5). The delivery system optics 128 scan the beam 126 over the corneal tissue of eye according to instructions from a computer 114 (e.g., such as the computer control assembly 16 shown in FIGS. 1-5). The computer 114 generally scans the beam 126 over the eye E by changing the angular position (e.g., by pivoting) of one or more mirrors (e.g., the tracking mirror 72) using galvanometric motors, or any of a wide variety of alternative scanning mechanisms. Optionally, the computer 114 may direct a profiling of the beam 26 using one or more variable apertures.

The system 100 may include a plurality of sensors 116 that produce feedback signals from moveable mechanical and optical components, such as those described in European Patent Application Publication No. 628298, which is incorporated herein in entirety by reference. Tracking processors 115 may have one or more separate processing structures from the computer 114, or may be integrated into the computer 114 as a single processor or with a wide variety of distributed processing arrangements. The computer 114 also includes a tangible medium 121 embodying the methods of the present invention in a machine readable code. Suitable media include floppy disks, compact optical disks (CDs), removable hard disks, or the like. In other embodiments, the code may be downloaded from a communication modality such as the Internet, and stored as hardware, firmware, or software, or the like.

In response to signals provided by tracking the processors 115 and the sensors 116, and according to the treatment to be performed on the eye, the computer 114 transmits command signals to motor drivers 118 and to the laser 120. In response to these command signals, the motor drivers 118 produce signals to change an angular orientation of a first stage pivot system 122 and a second stage pivot system 124, and to operate the other components of the system 100, such as to vary a size of a variable diameter iris to correct myopia, to control the distance between a pair of parallel blades so as to vary a width of the laser beam 126, to rotate an angular orientation of the parallel blades and rectangular beam to correct astigmatism, and the like. The computer 114 can compensate for lateral movement of the eye E during a sculpting procedure by directing the motor driver 118 to reposition the beam 126 (typically by movement of the first and second stages 122, 124) so that the therapeutic pattern of laser energy (to be directed at the eye E) remains aligned with the eye during voluntary and/or involuntary movements of the eye.

In broad terms, the horizontal and vertical cameras 113 capture images of the eye E from along imaging paths which are offset from the treatment axis of beam 26. The cameras 113 (e.g., infrared sensitive charge couple devices (CCD)) generate image signals that are transmitted to the tracking processors 115. The tracking processors 115 calculate a position of a feature of the eye E, and transmit signals indicating the position to computer 114. These signals represent an absolute position of the feature relative to the laser system 100, a relative position of the feature, a size of the feature, and the like. In some embodiments, the positional information may include a velocity of the feature, an acceleration of the feature, or the like. If sufficient tracking system performance is desired for tracking more rapid involuntary saccadic movements of the eye, the cameras 113 may be high-sampling rate image capture devices (e.g., with a sampling rate of about 250 Hz or more).

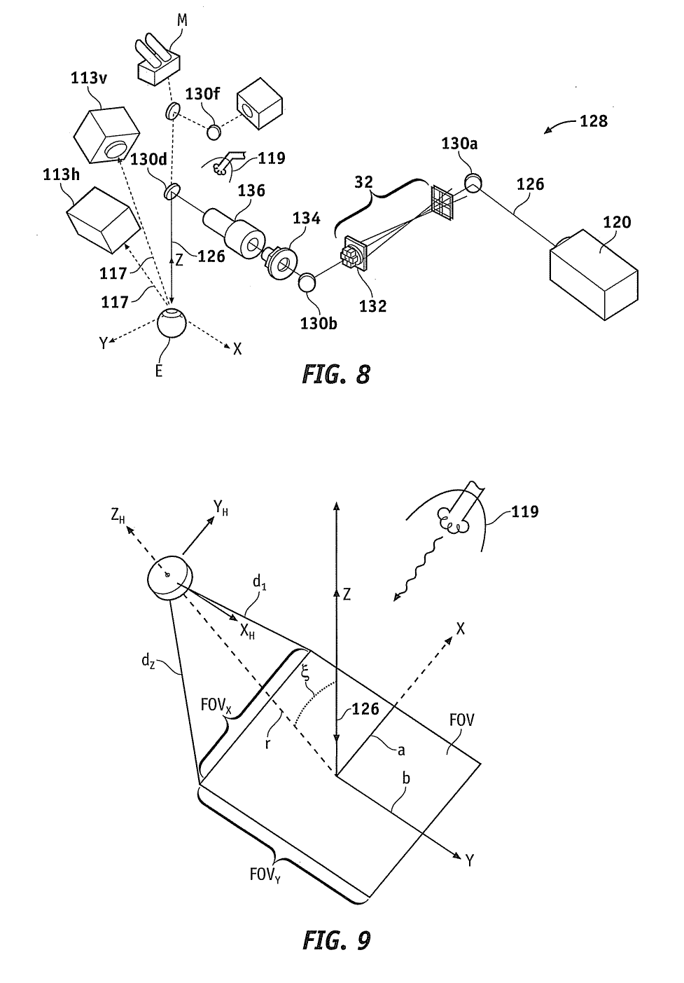

Typical delivery system optics 128 are illustrated without associated support structure in FIG. 8. Mirrors 130a and 130b (mirrors 130a, 130b . . . generally being referred to herein as mirrors 130) direct laser beam 126 through spatial and temporal integrators 132 and a variable aperture 134 prior to entering a scanning mechanism 136. The scanning mechanism 136 (which includes the first and second stages 122, 124 shown in FIG. 7) selectively deflects the beam 126 laterally across the corneal surface of eye E in the X-Y plane. While the system 100 is shown with a relatively large beam cross-section, the system 100 also provides advantages for a wide variety of laser eye surgery systems, including those having small-spot scanning lasers.

A variety of lenses may be provided for imaging, viewing the procedure using a microscope M, and the like. The trackers 111, 112 monitor movement of the eye E so that the computer 114 can compensate for the eye movement and accurately ablate the intended portion of the treatment area. A particularly advantageous eye tracker camera/processor is commercially available from ISCAN, INC. of Burlington, Mass. Ideally, the trackers 111, 112 are suitable for integration into VISX STAR® and VISX STAR S2® laser eye surgery systems, both which are commercially available from VISX, Inc. of Santa Clara, Calif. Embodiments of the system may also be incorporated into a variety other laser systems.

The laser 120 may include, but is not limited to, an excimer laser such as an argon-fluoride excimer laser producing laser energy with a wavelength of about 193 nm. Alternative laser systems may include solid state lasers, such as frequency multiplied solid state lasers, flash-lamp and diode pumped solid state lasers, and the like. Exemplary solid state lasers include UV solid state lasers producing wavelengths of approximately 188-240 nm such as those disclosed in U.S. Pat. Nos. 5,144,630, and 5,742,626; and in Borsuztky et al., Tunable UV Radiation at Short Wavelengths (188-240 nm) Generated by Frequency Mixing in Lithium Borate, Appl. Phys. 61:529-532 (1995). A variety of alternative lasers might also be used. The laser energy is generally a beam formed as a series of discreet laser pulses, and the pulses may be separated into a plurality of beamlets.

FIG. 8 also illustrates the position and orientation of horizontal and vertical cameras 113h, 113v. The horizontal camera 113h primarily measures movement of eye E along the X axis of the eye, and is positioned along the Y-Z plane and offset from the X-Z plane. The vertical camera 113v primarily measures movement of eye E along the Y axis, and is disposed along the X-Z plane and offset from the Y-Z plane, as illustrated. The horizontal and vertical cameras 113h, 113v are oriented toward the eye E along optical image paths 117 centered within the fields of view of the cameras 113h, 113v, and these optical paths 117 are generally defined by lenses of the associated camera structures. Each of the cameras 113 has an integrated analyzer for filtering light having a pre-determined polarization state. In another embodiment, the analyzers may be a separate component of the system 110 that is selectively interposed along the optical paths 117 to remove light having the pre-determined polarization state from the optical paths 117.

The horizontal and vertical cameras 113h, 113v, together with the tracking processors 115, may be commercially available tracking systems, such as those available from ISCAN, Inc. of Burlington, Mass., or other comparable systems. Suitable trackers generally include a position sensor and a processor for generating a position signal in response to signals from the sensor. Preferred trackers typically include a two-dimensional optical position sensor, often with optics for imaging the eye onto the sensor. An exemplary tracking system includes both an infrared CCD camera and a personal computer interface (PCI) card, together with software drivers compatible with an operating system running on the computer 114. Alternative camera structures having larger and/or smaller dimensions may be powered by a variety of sources, and may sense light in the visible or other wavelength ranges. As described above, the camera 113 provides an image signal to an associated tracking processor 115.

In use, the eye E is illuminated with a polarized infrared illumination source 19. The polarized infrared source 19 includes one or more infrared light-emitting diodes (LEDs) and a polarizer to produce light having the pre-determined polarization state. In an exemplary embodiment, lighting is provided by two banks of three infrared LEDs each, with each LED consuming about 80 mA. These banks of light-emitting diodes may be selectively energizable, with one bank of LEDs being energized when the right eye is aligned with a treatment axis of the system 100, and the other bank being energized when the left eye is aligned with the treatment axis. The LEDs are typically within about 90° (longitude) from the cameras 113 and are preferably angularly displaced (e.g., from the Z-axis) to a greater extent (e.g., have a larger azimuth angle such as a latitude from vertical) than the cameras 113.

Under polarized infrared illumination provided by infrared source 19, the pupil of eye E appears relatively dark to the cameras 113, as the infrared energy is not directly reflected by this clear structure. The area surrounding the pupil, including both the iris and sclera, presents a much lighter image to the cameras 113 under the infrared illumination, thereby producing a high contrast image of the pupil for tracking. Additionally, using the polarized illumination, specular reflections from the eye are removed by the analyzers from the resulting image captured by the cameras 113.

Because ambient lighting of the eye E may change during a procedure, the size of the tracked pupil may also change. To accommodate the changing size of the pupil, dynamic thresholding is a highly advantageous feature of the exemplary commercially available tracking camera. Dynamic thresholding is achieved by determining the pupil size while adjusting the threshold.

As described above, the scanning mechanism 136 laterally deflects the beam 126 in response to movement of the eye E as sensed by the cameras 113, such as described in U.S. Pat. No. 6,322,216, the entire disclosure of which is incorporate herein. The computer 114 (shown in FIG. 7) calculates the desired angular position of the first and second stages based in part on the location of the pupil sensed by horizontal and vertical cameras 113h, 113v. The computer 114 preferably determines a position of the pupil relative to the optical axis of the eye and/or of the laser delivery system using calculations which can be understood with reference to FIG. 9. These calculations are shown for the horizontal camera 113h, which is illustrated here schematically by an imaging lens of the camera. It should be understood that the vertical camera 113v may make use of similar calculations, except that the vertical camera 113v will be located at a position 90° offset from the horizontal camera 113h about the Z-axis, the optical axis of the eye E, and/or treatment axis of the laser beam 26. The horizontal axis, X_{H}, of the camera 113h is aligned along the X-axis. To minimize distortion along this desired measurement axis while providing an enhanced contrast, the horizontal camera 113h is disposed along the X-Z plane and is offset from the Y-Z plane by an angle ξ. ξ may be in a range from about 10° to about 70°, often being between about 15° and about 65°, preferably being between about 20° and about 50°, and more preferably being between about 25° and about 45°, ξ being about 27° in an exemplary embodiment.

The horizontal camera 113h images a region or field of view (FOV) of the eye E. The FOV is substantially rectangular in shape with a width indicated by FOV_{y} and a height indicated by FOVₓ. The eye surface region imaged within this field of view is generally at an angle of ξ relative to the camera 133h. A center 170 of the field of view is separated from camera 113h by a distance r, the top edge of the field of view is separated from the center 170 by a distance a, and the side edge of the field of view is separated from the center 170 by a distance b. The corners of the field of view FOV are separated from camera 113h by distances dᵢ, with i being 1, 2, . . . . As the field of view is generally symmetric about the Y-Z plane, the two distances of interest are d₁ and d₂, as illustrated.

Where x is the coordinate of the pupil along the X-axis of the coordinate system of the eye, and y is the coordinate of the pupil center along the Y-axis of the eye's coordinate system, the correct scaling factor is determined for the Y-component in the horizontal camera 113h and the X-component in the vertical camera 113v. The scale factor is used to calculate the y value, such as described in U.S. Pat. No. 6,322,216. Calculation of the x value follows a similar analysis, in which the scaling factor is determined and in which x is calculated using this scaling factor. As both the horizontal and vertical cameras 113h, 113v provide two-dimensional information at an angle to the treatment axis Z, the position of the eye E along the treatment axis Z may also be calculated.

Tracking of the eye E is preferably relative. In other words, when the system operator initiates tracking, the tracker 111, 112 records a position of pupil P as an initial position PO having a reference center location O. Subsequent eye movement is tracked relative to this central reference position O. Hence, absolute alignment of the tracker is not critical. However, tracking benefits significantly from accurate rotational alignment of the tracker components, as rotational misalignment may be more difficult and/or impossible to compensate for using software and the like.

The images provided by the two cameras 113h, 113v are processed by their associated PCI cards to determine a centroid of the pupil in the horizontal and vertical orientations. The pupil centroid data is available to the processor and/or processors of the system 10 when the tracker software triggers an interrupt. A datastream from the cameras 113h, 113v may contain duplicates as both horizontal and vertical data may be generated from each camera whenever either camera triggers a new image interrupt. A program may be used to remove duplicate data and maintain alignment to the data from the two cameras 113h, 113v. Optionally, this duplicate data may be used to verify that both trackers are operating within a predetermined tolerance, and/or to determine a vertical position of the pupil, as described above. If the trackers 113h, 113v appear to out of tolerance or if the patient's eye moves horizontally and/or vertically beyond a limited tracking/treatment zone, treatment may be interrupted. Timing information and the most recent pupil position are generally available to system programming via a data request/interrupt at all times.

In general, both a threshold level or value and a gated area can be determined to facilitate tracking of the pupil. The gated area generally includes a limited region of interest (ROI) within the image, and the exemplary gated area includes a rectangle within the image. Pixels inside the gated area are candidates for inclusion in the pupil, while pixels outside the gated area are excluded from potential inclusion within the pupil. The gated area is preferably selected to be as large as possible, while excluding unwanted edge material or features, such as a Lasik flap, eyelid, flap protector, speculum, or the like. The use of such a gated area helps to eliminate undesired artifacts near the edges of the field of view, but may also cause distortion as the pupil crosses the gated area boundary. Each tracking system applies a variety of tests before accepting a pupil position as valid, including a minimum separation between a pupil centroid and a gated area boundary, and the like. If any of these tests are not fulfilled, a tracking error condition may be identified, and a tracking error signal may be generated.

Each time a system operator initiates a treatment with the laser eye surgery system 100, the application may “dynamically threshold” or generate a pupil threshold level automatically. In one embodiment, this can be accomplished by acquiring a number of separate images at differing illumination threshold settings. Pupil size may be calculated for each of these differing images, and the pupil sizes may be analyzed as a function of threshold setting. The threshold/pupil size curve has a characteristic shape in which the gradient of the curves is generally below a predetermined or assigned value between two points. The gradient generally increases beyond these two points along the curve, and the optimum threshold value is somewhere between these points on a relatively flat part of the curve.

A typical laser surgery procedure proceeds with the system operator positioning the patient while the trackers 111, 112 are off. The system operator positions the laser delivery optics 128 relative to the patient's eye E with the horizontal and vertical cameras 113, 113v mounted relative to the delivery system optics 128 to be aligned with the eye E. The microscope M is focused on the eye E, and the trackers 111, 112 are enabled by the system operator inputting a command to the system 100, typically by pressing a keypad button.

The system operator aligns the eye E (e.g., with a reticle of the microscope M) to establish the reference position of the tracker 111, 112. Once the eye is aligned, the system operator provides another input command, such as by pressing a foot switch. The pupil position at the time of this second input command O becomes the tracker origin.

The tracker 111, 112 thereafter gives movement coordinate vectors to the system 100 from the tracker origin. In many embodiments, an indication will be displayed to the operator, optionally as a light within the field of view of the microscope M to show that tracking is operative. The eye tracker 111, 112 generally remains “on” until another input command from the system operator, such as again pressing the keypad button, with the button toggling the tracker between “on” and “off.”

If tracking is lost during a treatment (e.g., while the system operator intends to maintain a treatment by continuing to depress a foot pedal), a loss of tracking indication may be provided to the system operator, such as by a flashing indicator within the microscope M or on any other system display. Optionally, laser sculpting may be automatically interrupted if tracking is lost. If the procedure is interrupted prior to completion (in many laser eye surgery system, by partially releasing a foot pedal), the tracker may keep the stored reference position until and/or unless the procedure is fully interrupted by fully releasing the foot pedal, or the like.

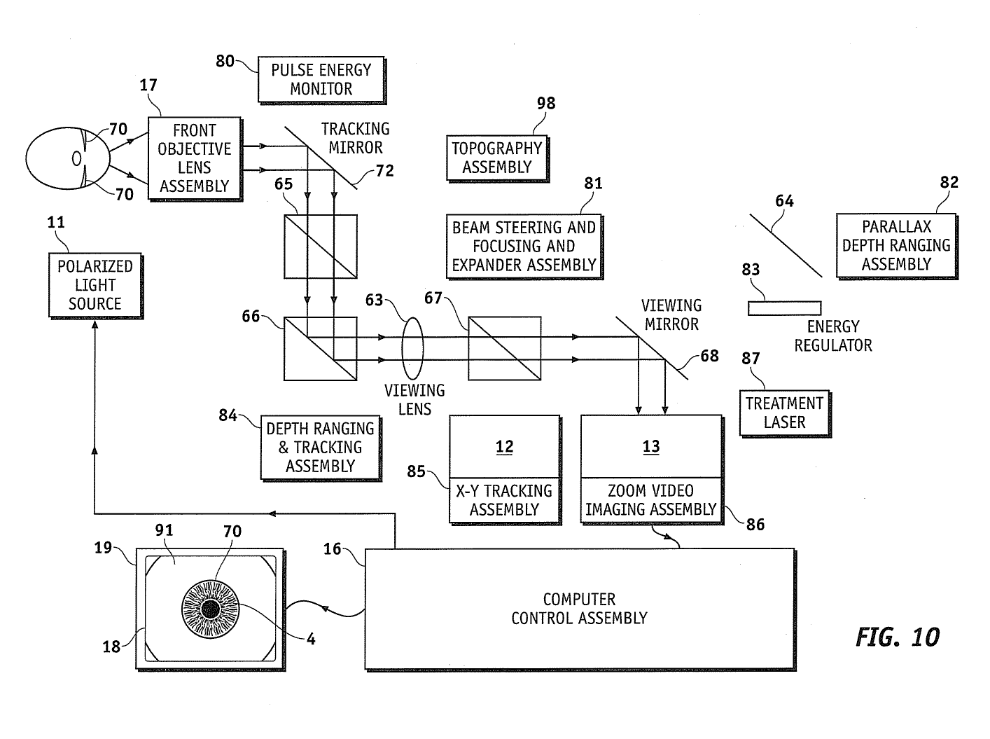

FIG. 10 shows an imaging assembly control loop. The imaging assembly 86 includes a low-level-light camera and zoom optics. The camera preferably is an intensified video camera, for example a silicon intensified target (SIT) tube camera. In other embodiments, the camera can be a conventional video camera in combination with a microchannel-plate intensifier. The camera sensitivity is preferably about 1000 times the sensitivity associated with a normal video camera, enabling the system 10 to look at weakly scattered light and targets that are poorly illuminated for the desired levels of high magnification at greater working distances.

In one embodiment of the present invention, the system 10 uses a combination of specular and scattered light techniques for detecting and identifying diffusely reflecting surfaces, specularly reflecting surfaces, surface displacements, features, and shapes of the patient's tissue. This is particularly useful in the eye where differentiating between the amorphous tear layer anterior to the cornea and the structured epithelial surface layer of the cornea is difficult. Even the cell walls of the endothelial cells of the cornea or of the anterior lens capsule tend to scatter light. The imaging assembly 86 can produce an image of these actual cells by forming an image composed of detected scattered light. The imaging assembly 86, as well as the tracking camera, can substantially exclude specularly reflected light by cross polarization of selectively polarized illuminators. Other methods for reducing specular reflections preferentially to scattered images are also possible.

In one embodiment, the optics of the imaging assembly 86 provide flat field, anastigmatic, achromatic, nearly diffraction limited imaging with optical magnification zoomable approximately over a 15-fold range of about 15× to about 200×. This magnification is adjustable and is typically selected to correspond to the largest magnification that can still be comfortably used for situating a lesion (e.g., the smallest field of view that can be used when magnified across the fixed display size of a video monitor 18 of the user interface 19). For example, for corneal refractive surgery, where the surgeon observes the cornea from limbus to limbus, a field of view of approximately 12 to 14 mm is used. At the screen, the zoom optics allow for adjustable magnification in the range of about 15× to about 200×, for example. This enables the surgeon to view a very narrow field (e.g., on the order of a millimeter in width) or a much wider field at lesser magnification. This is useful in confirming aim and focus at a desired region. Zooming can be effected through use of a joystick, trackball, mouse, or other pointing device accessing a scroll bar in the user interface 19. The viewing mirror 68 can shift the image on the monitor 18 to the left or right or up or down, independent of the aiming of any other subsystem.

An ablation profile can be generated based on a wavefront measurement of the patient's eye. For example, Hartmann-Shack sensors, or the like, can be used to obtain a wavefront elevation surface of the patient's eye. The wavefront elevation surface can then be processed by a treatment algorithm to generate a treatment table or ablation profile that is customized to correspond to the patient's wavefront elevation surface. The components of one embodiment of a wavefront system for measuring the eye and ablations comprise elements of a VISX WaveScan®, available from VISX, Inc. of Santa Clara, Calif., at least some of which may be incorporated into the system 10. An alternate embodiment of a wavefront measuring device is described in U.S. Pat. No. 6,271,915, the entire disclosure of which is incorporated herein by reference.

A treatment program map may be calculated from the wavefront elevation map to remove the regular (e.g., spherical and/or cylindrical) and irregular errors of the optical tissues. By combining the treatment program with a laser ablation pulse characteristics of a particular laser system, a table of ablation pulse locations, sizes, shapes, and/or numbers can be developed. One example of a method and system for preparing such an ablation table is described in U.S. Pat. No. 6,673,062, the entire disclosure of which is incorporated herein by reference. The ablation table may optionally be optimized by sorting the individual pulses to avoid localized heating, minimize irregular ablations if the treatment program is interrupted, and the like.

Referring back to FIG. 1, the wavefront data and/or the customized ablation profile can be provided to the system 10 through reading of a computer readable medium or through delivery into a memory of the system 10 over a local or wide-area network (LAN or WAN). The computer control assembly 16 can have software stored in a memory and hardware that can be used to control the delivery of the ablative energy to the patient's eye, the tracking of the position (translations in the x, y, and z directions and torsional rotations) of the patient's eye relative to an optical axis of laser beam, and the like. In exemplary embodiments, among other functions, the computer control assembly 16 can be programmed to calculate the customized ablation profile based on the wavefront data, register the reference image(s) with the image(s) captured by the imaging assembly 86, and measure the torsional offset, between the patient's eye in the two images. Additionally, the computer control assembly 16 can be programmed to measure, in real-time, the movement (x(t), y(t), z(t), and rotational orientation .theta.(t)) of the patient's eye relative to the optical axis of the laser beam so as to allow the system 10 to modify the delivery of the customized ablation profile based on the real-time position of the patient's eye.

In order to register an ablation profile and the patient's eye during the laser treatment, the ablation pattern and the patient's eye should share a common coordinate system. Thus, the ablation profile should be positionally and torsionally aligned with the patient's eye when the patient's eye is positioned in the path of the laser beam. Additionally, the translational and torsional orientation of the patient's eye should be tracked during the surgical procedure to ensure an accurate delivery of the ablation profile.

During the calculation of the wavefront elevation surface, a reference image or iris image of the patient's eye is obtained. The image of the patient's eye can be analyzed by an algorithm that locates the center of the pupil and/or iris, calculates the radius of the pupil and/or iris, and locates markers in the patient's iris for subsequent registration and tracking. To torsionally align (i.e., register) the ablation profile with the patient's eye, the coordinates of the reference or iris image of the eye are transformed to an image of the eye (e.g., captured by the imaging assembly 86 shown in FIGS. 1-5) so as to determine the positional differences and torsional offset between the two images of the eye, θ₀. In one embodiment, the imaging assembly 86 is a video device that can obtain streaming video of the patient's eye and operate as a torsional tracker. One frame of the streaming video, typically the first frame of the streaming video, can be analyzed by the computer control assembly 16 to locate the pupil center, iris center, and/or markers that were originally located in the reference image. Once the pupil center, iris center, and/or markers are located, a torsional offset, θ₀, between the reference image and video frame image of the patient's eye is calculated.

The polarized light source 11 may be used to illuminate the eye with light having the pre-determined polarization state, as previously mentioned herein. In this embodiment, the imaging assembly 86 includes an analyzer 13 for filtering light having the pre-determined polarization state. In another embodiment, the analyzer may be a separate component of the system 10 that is selectively interposed along the optical path between the viewing mirror 68 and the imaging assembly 86 to remove light having the pre-determined polarization state from the image captured by the imaging assembly 86. Thus, specular reflections can be removed from the images captured by imaging assembly 86 that are utilized with torsional tracking and registration.

After the torsional offset θ₀ is determined, the computer control assembly 16 can track the translational position (x(t), y(t), and z(t)) of the patient's eye with a high speed eye tracker (HSET) (e.g., the X-Y tracking assembly 85 and the depth ranging and tracking assembly 84) and the torsional orientation (θ(t)) of the eye with the torsional tracker. Because the position of the center of the pupil is tracked with the HSET, the torsional tracker generally has to estimate the position of the markers with respect to the pupil center.

If the HSET determines that the patient's eye has moved (relative to the video frame image), the computer control assembly 16 can correct the delivery of the customized ablation pattern by adjusting the patient's customized treatment table. For example, the translation and torsional measurements may be added into the treatment table. To track the torsional movement of the patient's eye, the torsional tracker can use the markers identified above, other high-contrast iris patches, or if the patient's iris contains too little texture, the surgeon has the option of creating artificial landmarks on the eye for tracking. In some embodiments, the algorithm determines if artificial markers are required. The translational position and torsional orientation of the patient's eye are preferably tracked and analyzed in real-time so that the x(t), y(t), z(t) and θ(t) information can be used to adjust the customized treatment table and so that the laser 87 delivers the appropriate ablation pattern to the patient's eye. Examples of systems and methods for tracking a torsional orientation and position of an eye are described in U.S. Pat. No. 7,044,602, the entire disclosure of which is incorporated herein by reference.

In another embodiment, a time series of wavefront data readings may help to provide a more accurate overall determination of the ocular tissue aberrations. As the ocular tissues can vary in shape over a brief period of time, a plurality of temporally separated wavefront sensor measurements can avoid relying on a single snapshot of the optical characteristics as the basis for a refractive correcting procedure. Still further embodiments are also available, including obtaining wavefront sensor data of the eye with the eye in differing configurations, positions, and/or orientations. For example, a patient often assists in maintaining alignment of the eye by focusing on a fixation target, as described in U.S. Pat. No. 6,004,313, the entire disclosure of which is incorporated herein by reference. By varying a focal position of the fixation target as described in that reference, optical characteristics of the eye may be determined while the eye accommodates or adapts to image a field of view at a varying distance. Further embodiments include rotating the eye by providing alternative and/or moving fixation targets.

As described above, a first step entails registering a reference image of the eye taken during the calculation of the wavefront elevation map with a second image of the eye taken just prior to the delivery of the ablation energy. To torsionally register a reference image with a second image of the eye (e.g., to determine the torsional displacement between the two images of the eye), an initial step is to obtain the first or reference image. In most configurations, the smallest distance between the edge of the pupil and the obstructing elements, such as eyelids, eyelashes, strong shadows or highlights should be sufficiently large to leave a portion of the iris completely exposed for the entire 360° range. The largest possible portion of the iris is preferably in sharp focus to expose texture.

A pupil finding algorithm can be used to locate the pupil, calculate the radius of the pupil, and find the center of the pupil. In one embodiment, the pupil is located using threshold evaluation of the image. For example, by analyzing a pixel value histogram of the image, the position of a first “dip” in the histogram is choosen after at least 2000 pixels are below a pre-determined cutoff threshold. All pixels below the threshold are labeled with “1” and pixels above the threshold are labeled with “0”. Pixels labeled with “1” generally correspond to the pupil, eyelashes, and possibly other regions of the image. The number of pixels employed may vary with the area of the pupil.

The relatively large size and central location of the pupil region provide two distinguishing features, compared to other non-pupil regions. In some embodiments, regions intersecting with a five-pixel wide inner frame of the image can be discarded and the largest remaining region can be selected as the pupil. If desired, the selected pupil region can be filled to remove any holes created by reflections, or the like. For example, in one embodiment, the remaining region of the image may also be analyzed for convexity. If the ratio of the area of the region to the area of a corresponding convex hull is less then about 0.97, a circle completion procedure can be applied to the convex points on the boundary of the region. A radius and center of the pupil can be estimated by a standard weighted least-square estimation procedure. If the convexity quotient is above about 0.97, the radius and centroid can be obtained using conventional methods.

Optionally, in some embodiments an iris finding algorithm can be used to locate the iris, calculate the radius of the iris, and/or locate the iris center. Since the images of the eye (e.g., the reference image and the image captured by the imaging assembly 86) both contain the pupil and iris, the images may be registered by calculating the center of the pupil and the center of the iris and expressing the position of the pupil center with respect to the center of the iris. The center of the iris may be described as a center of a circle corresponding to the outer boundary of the iris. The position of the center of the iris can be used to calculate a pupil offset from the iris center. Even if the iris or pupil are not circular (e.g., elliptical), a center can be determined for each of the pupil and iris.

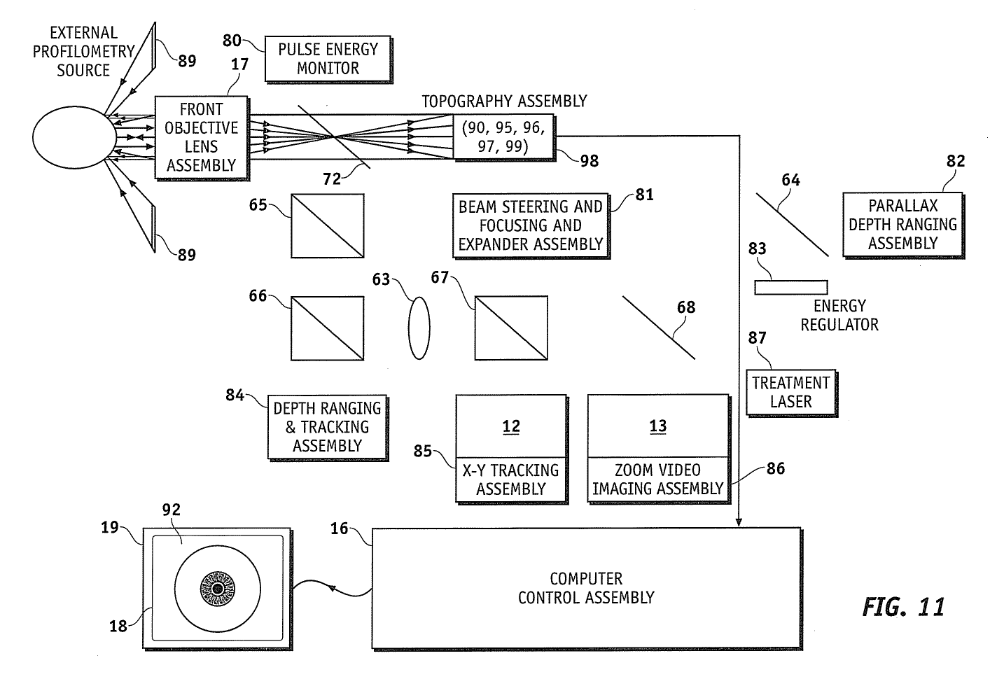

FIG. 11 shows the light path for the topography assembly 98. In this embodiment, the topography assembly 98 provides a three-dimensional mapping system of the surface of the target (e.g., the eye of the patient). In one embodiment of the system 10 (such as described by U.S. Pat. No. 5,054,907 and U.S. Pat. No. 5,170,193, all of which are incorporated herein by reference), the topography assembly 98 includes a light projector 95 including an internal profilometry source 90, an illumination mask 96, an optical collection system 94, and a profilometry assembly. The profilometry assembly includes an adjustable aperture 99 and a CCD camera 97 equipped with a frame grabber, such as an array of dots arranged into rings and radial spokes converging to a common center, onto the tear layer of the eye. The reflected images of the predetermined pattern are collected by the optical assembly 94, which may include a set of plates to correct for any astigmatism induce by the tracking mirror 72 and any other interior mirrors, fed to the camera 97 through the aperture 99 for analysis.

By controlling the angle of acceptance of the light bundle from each virtual image, the adjustable aperture 99 acts as a spatial filter, providing a physical representation of the source of paraxial rays through trade-offs between resolution and brightness. The camera 97 includes means to digitize and electronically enhance the images. The signals are fed to a microprocessor that performs preliminary displacement analysis using software means (e.g., embedded within the computer control assembly 16) based on mathematical morphological transformations, such as described by U.S. Pat. No. 5,170,193. The transformations include a solution of a set of coupled differential equations, whereby the local normals and curvature parameters are computed at each data point so that a surface can be computed to within the measurement accuracy, and subsequently displayed on the video monitor 18. The methods of light projection and profilometry permit the system 10 to operate with low intensity light signals to enhance safety and patient comfort while extracting significant signal levels from the noise background.

In other embodiments of the profilometry assembly, alternative projection techniques may be utilized in place of or in addition to the mapping and projection means described above. In one embodiment, an external profilometry source 89 (e.g., an array of LEDs) projects a pattern of dots onto the eye in a manner described by U.S. Pat. No. 5,283,598. In this embodiment, curvature measurements of the anterior surface of the cornea can be obtained radially extending out to about 8 mm. Other techniques based on off-axis illumination may utilize, for example, a slit lamp illuminator to obtain measurements of the thickness of the cornea, the depth of the anterior chamber, and/or the thickness of the lens (the latter coupled with standard keratoscopy methods to correct for corneal curvature). Mounting the slit lamp at a fixed location relative to a CCD camera (such as the camera 97) and rotating the entire structure around a central axis provides a method to collect global corneal data (e.g., out to the limbus) without sacrificing local accuracies, given the simultaneous 3-D tracking capability already contained in the system. In this manner, the domain of topographic measurements can be extended from limbus to limbus while providing pachymetry data as well. Topography methods based on Ronchi grating in conjunction with Moire interferometry, or advanced holographic techniques as discussed by e.g., Varner (in Holographic Nondestructive Testing, Academic Press, New York, 1974 pp. 105) and by Bores (in “Proceedings of Ophthalmic Technologies,” SPIE 1423, C. A. Puliafito, ed., pp. 28 (1991)), may be utilized in other embodiments of the system 10.

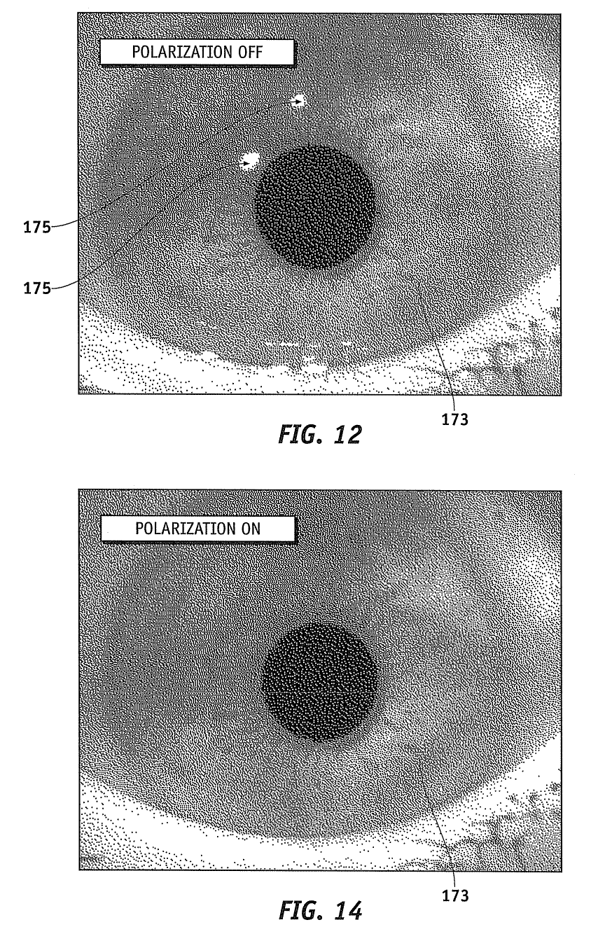

FIG. 12 is an image of an eye 173 without polarized illumination. The image is captured by an image capture device, such as a camera, eye tracker, or torsional tracker. Specular reflections 175 (e.g., from the tear film, epithelium, or the like) are present in the image. During ophthalmic surgery, the eye is illuminated with electromagnetic radiation (e.g., light) having a wavelength from about 100 nm to about 1500 nm. For eye imaging purposes (e.g., during tracking, position detection, registration, and the like), light having a wavelength from about 400 nm to about 1000 nm are commonly used. For purposes of eye alignment, common natural eye features are typically detected, such as the pupil, limbus, iris structure, blood vessels, and the like. Eye alignment may also be based on artificially created features, such as ink marks placed on the eye by the surgeon, incisions associated with a LASIK flap cut, and the like. Image formation is based on illuminating the eye and detecting backscattered light from the eye.

Light rays that move from a medium of a given index of refraction into another medium with a different index of refraction experience both reflection and refraction. Fresnel equations generally govern the intensity of the reflected light. If the polarization of the illuminating beam is parallel to the surface of the interface (e.g., between the two media having different indices of refraction), a specific angle exists where the polarized light is fully transmitted and not reflected. For all other angles, a portion of the incident beam is reflected at the interface with preserved polarization orientation. The refracted beam experiences multiple refractive index discontinuities causing scatter, attenuation, and random polarization (e.g., diffuse reflection).

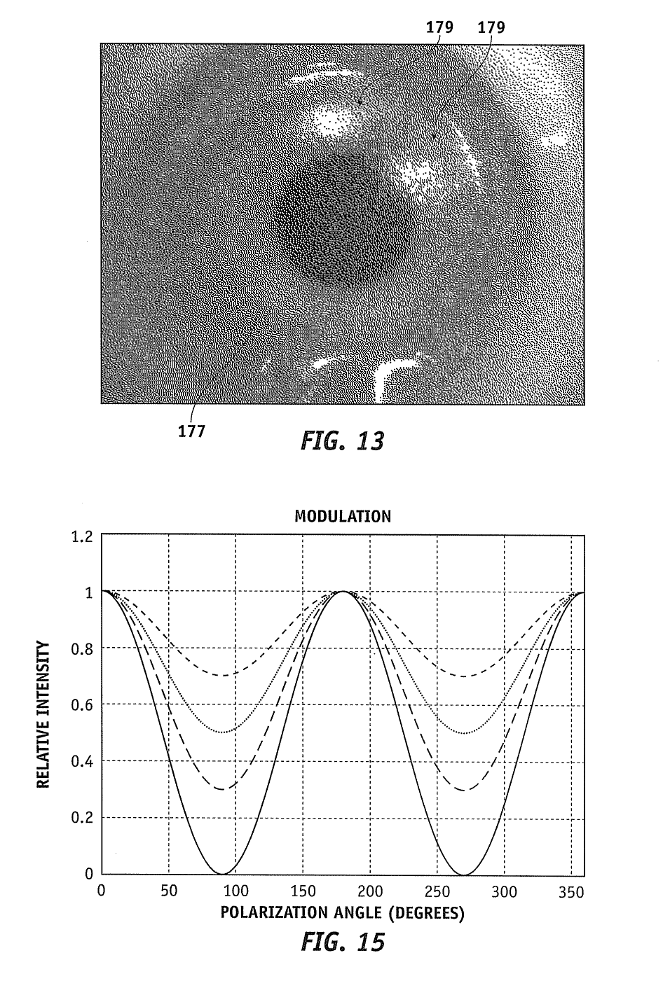

During refractive surgery, the process of creating a corneal flap (e.g., to reveal a stromal bed for receiving laser treatment) introduces refractive index discontinuities in the bulk cornea and also results in increased surface roughness. A rough surface typically shows an increased amount of specular reflection as more surface elements behave as a mirror-like surface. FIG. 13 is an image of a corneal flap bed 179 of an eye 177. The corneal flap bed is a source of specular reflections 179.

FIG. 14 is an image of the eye 173 shown in FIG. 12 using polarized illumination (e.g., from the polarized light source 11) and an analyzer. The analyzer filters the light received by the image capture device (e.g., the aforementioned camera, eye tracker, or torsional tracker). Specular reflections generally maintain the polarization state of the polarized illumination. Using the analyzer (e.g., the analyzer 12, 13) to filter light having the same polarization state, the specular reflections 175 (shown in FIG. 12) are removed. The same technique may be used to remove the specular reflections 179 in the image of the corneal flap bed 179 (shown in FIG. 13).

Additionally, various layers associated with different structures of the eye can be characterized by a relationship of a measured reflectivity versus a pre-determined polarization state of radiation (e.g., light) impinging on the eye (e.g., the polarization state of the polarized illumination). FIG. 15 is a graph of multiple waveforms 181, 183, 185, 187 illustrating relationships between the relative intensity of specular reflections and the incident polarization angle (e.g., the polarization orientation of the analyzer). Each of the waveforms 181, 183, 185, 187 corresponds to a different polarization orientation of the illuminating light. Based on the polarization orientation of the illuminating light, the relative intensity of specular reflection generally decreases from a first waveform 181 to a second waveform 183 to a third waveform 185 and to a fourth waveform 187. A rapid and distinct change in slope of this relationship (e.g., at about 90° or at about 270°) at the characteristic polarization state can be used to identify the corresponding different structures of the eye. At these angular orientations (e.g., at about 90° or at about 270°), the removal of specular reflections is maximized with respect to the analyzer. Additionally, the polarization orientation of the illuminating light (e.g., in this example, the waveform 187) is selected to maximize the degree of specular reflection removal (e.g., based on the minimizing the relative intensity of specular reflections). Thus, the anterior surface layer of the cornea and sclera, the sub-epithelial treatment layer, the posterior layer of the cornea, the iris layer, the anterior layer of the lens, the posterior layer of the lens, and the retina layer are all layers that may be characterized by a relationship of the measured reflectivity versus the polarization state of the light illuminating the eye. The light reflected from the eye can be filtered (e.g., via the analyzer) and analyzed for rapid and distinct changes in the slope. Each of these slope changes (e.g. minima) can be associated with one of the aforementioned layers.

While embodiments of this invention have been shown and described, it will be apparent to those skilled in the art that many more modifications are possible without departing from the inventive concepts herein.

What is claimed is:

1. A system for sensing a movement of an eye, the system comprising:
a light source configured to illuminate the eye with a first light having a polarization state;
an image capture apparatus configured to generate at least one image of the eye based on a reflected light from the eye, the reflected light being based on the first light;
an analyzer optically coupled to the image capture apparatus and configured to filter a second light from the reflected light, the second light having the polarization state; and
a processor coupled to the image capture apparatus and configured to determine the movement of the eye based on the at least one image of the eye.

2. A system according to claim 1, wherein the eye has a surface, wherein the first light has a lower coherence length and has a polarization vector in parallel with the surface of the eye, and wherein the analyzer is oriented about 90 degrees from the polarization vector.

3. A system according to claim 1, wherein the eye has a stromal bed, wherein the light source is further configured to illuminate the stromal bed with the first light, and wherein the analyzer is further configured to filter the second light from the reflected light from the stromal bed.

4. A system according to claim 1, wherein the eye has an optical axis and first and second lateral axes, wherein the image capture apparatus comprises:
a first tracker oriented toward the eye along a first axis and configured to generate a first image of the eye, the first axis angularly offset from the optical axis; and
a second tracker oriented toward the eye along a second axis and configured to generate a second image of the eye, the second axis angularly offset from the optical axis;
wherein the analyzer is further configured to:
filter the second light from a first reflected light from the eye along the first axis, the first reflected light being based on the light beam; and
filter the second light from a second reflected light from the eye along the second axis, the second reflected light being based on the light beam; and
wherein the processor is further configured to:
determine a first movement of the eye relative to the first lateral axis based on the first image; and
determine a second movement of the eye relative to the second lateral axis based on the second image.

5. A system according to claim 1, wherein the eye has an optical axis and first and second lateral axes, wherein the image capture apparatus comprises:
a first tracker oriented toward the eye along a first axis and configured to generate a first image of the eye, the first axis angularly offset from the optical axis; and
a second tracker oriented toward the eye along a second axis and configured to generate a second image of the eye, the second axis coaxial with the optical axis;
wherein the analyzer is further configured to:
filter the second light from a first reflected light from the eye along the first axis, the first reflected light being based on the light beam; and
filter the second light from a second reflected light from the eye along the second axis, the second reflected light being based on the light beam; and
wherein the processor is further configured to:
determine a first movement of the eye relative to the first lateral axis based on the first image; and
determine a second movement of the eye relative to the optical axis based on the second image.

6. A system according to claim 1, wherein the image capture apparatus is further configured to generate first and second images of the eye, and wherein the processor is further configured to:
determine a common reference point in the first and second images of the eye;
locate at least one marker in an iris of the first image;
find at least one corresponding marker in the second image; and
correlate an orientation of the at least one marker in the first image with an orientation of the at least one corresponding marker in the second image; and
cyclotorsionally register the first and second images by substantially translationally matching the common reference point and the at least one marker of the first and second images.

7. A system for ablating a cornea of an eye with a laser treatment, the system comprising:
a laser assembly configured to output a pulsed laser beam;
an image capture apparatus comprising a light source configured to illuminate the eye with a first light having a polarization state, the image capture apparatus configured to generate at least one image of the eye based on a reflected light from the eye, the reflected light being based on the first light;
an analyzer optically coupled to the image capture apparatus and configured to filter a second light from the reflected light, the second light having the polarization state; and
a controller coupled to the laser assembly and the image capture apparatus, the controller configured to:
determine a movement of the eye based on the at least one image of the eye; and
direct the laser assembly to deflect the pulsed laser beam in correlation with the movement of the eye.

8. A system according to claim 7, wherein the eye has first and second lateral axes, the first lateral axis orthogonal to the second lateral axis, and wherein the movement of the eye is selected from the group consisting of a first movement of the eye along the first lateral axis and a second movement of the eye along the second lateral axis.

9. A system for ablating a cornea of an eye with a laser treatment, the laser treatment generated in association with a first image of the eye, the system comprising:
a laser assembly configured to output a pulsed laser beam;
an image capture apparatus comprising a light source configured to illuminate the eye with a first light having a polarization state, the image capture apparatus configured to generate a second image of the eye based on a reflected light from the eye, the reflected light being based on the first light;
an analyzer optically coupled to the image capture apparatus and configured to filter a second light from the reflected light, the second light have the polarization state; and
a controller coupled to the laser assembly and the image capture apparatus, the controller configured to:
register the first image and the second image;
align the laser treatment with the second image of the eye; and
direct the laser assembly to output the pulsed laser beam at the cornea in correlation with the laser treatment.

10. A system according to claim 9, wherein the controller is further configured to center and torsionally align the laser treatment with the second image of the eye.

11. A method of modifying a refractive profile of an eye with a laser treatment, the method comprising the steps of:
illuminating the eye with a first light having a polarized state;
filtering a second light from a reflected light from the eye, the reflected light based on the first light, the second light having the polarized state;
capturing at least one image of the eye based on the reflected light from the eye;
determining a position of the eye based on the at least one image of the eye;
aligning a laser treatment based on the position of the eye; and
directing a pulsed laser beam at a corneal surface of the eye in correlation with the laser treatment.

12. A method for measuring an eye throughout phases of an ophthalmic surgery, the eye comprising an anterior surface layer of a cornea and a sclera, a sub-epithelial treatment layer, a posterior corneal layer, an iris layer, an anterior lens layer, a posterior lens layer, and a retina layer, the layers being characterized by a relationship of a measured reflectivity versus a polarization state of the radiation impinging on the eye that exhibits a rapid and distinct change in slope at a characteristic polarization state, the method comprising the steps of:
generating one or more light beams having a polarization state;
directing the light beams at the eye;
separating pre-determined polarization states of light beams reflected from the anterior surface layer and one or more of the sub-epithelial treatment layer, the posterior corneal layer, the iris layer, the anterior lens layer, the posterior lens layer, and the retina layer; and
directing the separated light beams to one or more sensor elements to measure an intensity.

13. The method according to claim 12, wherein the sub-epithelial treatment layer is created by laser induced breakdown or mechanical cutting.

14. The method according to claim 12, wherein the anterior surface layer is created by one of laser ablation, chemically dissolving, mechanical scraping of the eye, and lifting a section of the cornea to expose the sub-epithelial treatment layer whereby the relationship of measured reflectivity versus polarization state of the radiation impinging on the eye is substantially altered.

15. The method according to claim 12, wherein portions of the anterior surface layer are sequentially removed to effect a change in the surface profile.

16. The method according to claim 12, wherein the wavelength of the one or more light beams is substantially outside of a visible spectral range from about 400 to about 700 nanometers, wherein the one or more light beams has an intensity below about 20 mW/cm² at the anterior surface layer and an intensity below about 0.7 W/cm² at the retina layer, and wherein the polarization state is selected from the group consisting of a linear polarization, an elliptical polarization, a circular polarization, and a random polarization.

17. The method according to claim 12, wherein the cornea and the lens are substantially transparent to the one or more light beams.

18. The method according to claim 12, wherein the one or more light beams has a plurality of wavelengths comprising both visible and near infrared wavelengths.

19. The method according to claim 12, wherein the one or more sensor elements are imaging sensors of cameras.

20. The method according to claim 12, wherein the one or more sensor elements are elements of a Shack-Hartmann wavefront sensor configured to receive the reflected light beams from the retina layer.

21. The method according to claim 12, wherein the one or more light beams are directed in a predetermined pattern to the anterior surface layer, and wherein the one or more sensor elements are elements of an imaging sensor used for corneal topography.

22. The method according to claim 12, wherein the separating step allows measuring the light beam using the one or more sensor elements with limited dynamic range.

23. The method according to claim 12, wherein the light beams reflected from subsurface layers have been reflected multiple times before reaching the subsurface layer.
